(19) United States
(12) Patent Application Publication
(10) Pub. No.: US 2012/0109195 A1
(43) Pub. Date: May 3, 2012

(54) ELASTOMERIC THREAD HAVING ANCHORING STRUCTURES FOR ANCHORING IN BIOLOGICAL TISSUES

(75) Inventors: Erich Odermatt, Schaffhausen (CH); Ingo Berndt, Tuttlingen (DE); Silke König, Rottweil (DE); Sven Oberhoffner, Weinstadt-Endersbach (DE); Erhard Müller, Stuttgart (DE)

(73) Assignees: ITV DENKENDORF PRODUKTSERVICE GMBH, Denkendorf (DE); AESCULAP AG, Tuttlingen/Donau (DE)

(21) Appl. No.: 13/318,809

(22) PCT Filed: May 10, 2010
(86) PCT No.: PCT/EP10/02848

§ 371 Date: Jan. 17, 2012

(30) Foreign Application Priority Data
May 8, 2009 (DE) 10 2009 020894.1

(51) Int. Cl. A61B 17/04

(52) U.S. Cl. 606/232

(57) ABSTRACT

An elastomeric thread for use as a knotless or self-fixating wound closure system includes on its surface anchoring structures for anchoring in biological, more particularly human and/or animal, tissues.

### RELATED APPLICATIONS

This is a §371 of International Application No. PCT/EP2010/002848, with an international filing date of May 10, 2010 (WO 2010/127875 A1, published Nov. 11, 2010), which is based on German Patent Application No. 10 2009 020 894.1, filed May 8, 2009, the subject matter of which is incorporated by reference.

### TECHNICAL FIELD

This disclosure relates to an elastomeric thread, a wound closure system based thereon, a surgical kit of parts, a process for producing the elastomeric thread and use of the elastomeric thread for producing a wound closure system.

### BACKGROUND

The standard way of closing wounds in surgery is by using thread-shaped sutures. These are typically knotted to achieve secure fixation in the tissue. Care must be taken to ensure that the wounds to be closed are stitched together using an optimal force at the wound edges. If, for example, the wound edges are stitched together too loosely and too nonuniformly, there is a risk in principle of increased scarring or dehiscence. If, by contrast, the wound edges are stitched together overly tautly, there is a risk that blood flow through the wound edges is restricted, which can give rise to necrotic changes in the surrounding tissue region.

In addition to the risk of possible secondary complications, which may necessitate renewed surgical interventions, there is also always a certain risk that wound closures based on knotted sutures will lead to disruptions in the healing process and more particularly to unsatisfactory cosmeses for the patient concerned. Another factor is that it is often necessary for several knots, more particularly up to seven knots, to be placed on top of each other to ensure a secure knotted hold. This means that there is a lot of material being introduced into the region of the wound to be cared for, and can lead to increased foreign-body reactions, more particularly in the case of absorbable sutures. Furthermore, the use of conventional sutures can also be of disadvantage in the management of difficult-to-access fields of indication, as typically occur in laparoscopy.

Sutures which, in contradistinction to known or conventional threads, do not have to be knotted have been known for some time under the designation “barbed sutures.” Such knotless or self-locking/self-fixating/self-retaining sutures usually consist of a monofil thread that has barbs along its longitudinal axis. Corresponding sutures are described, for example, in U.S. Pat. No. 3,123,077 A, EP 1 559 266 B1, EP 1 560 683 B1 and EP 1 555 946 B1. The barbs have generally been formed on the thread surface such that the thread can be pulled through the tissue in the direction of the barbs without great resistance and more particularly without significant tissue trauma. When pulled in the opposite direction, however, the barbs deploy and anchor themselves and, hence, also the suture in a surrounding tissue region. This prevents the suture being pulled back through a puncture channel formed by the suture.

It is a profound disadvantage that, as the knotless or self-fixating sutures described in the previous section are introduced into tissue, the barbs, which generally protrude from the thread surface, can give rise to additional irritation of the tissue. In addition, as well as tissue traumatization, the protruding barbs give rise to a larger puncture channel in the course of being pulled through the tissue. To avoid additional irritation or traumatization of tissue, therefore, appropriate auxiliary means are frequently employed. For instance, techniques are known from facial surgery where initially a thin tube is led through the tissue to be treated before a barbed suture is led through this tube. After correct placement of the suture, the tube is removed, which causes the barbs to become erect and, hence, able to effect an anchoring of the suture in the surrounding tissue region. There are other surgical methods where a barbed suture is pulled together with a flexible tube surrounding the suture through a tissue to be treated. Once the sheathed suture is correctly placed, the flexible tube is pulled off, making it possible for the suture to become anchored via its barbs. Appropriate auxiliary means and suture systems based thereon are described, for example, in U.S. Pat. No. 6,241,747 B1 and DE 10 2005 004 318 A1. The use of additional auxiliary means makes the above-described methods more costly, inconvenient and complicated to carry out and thereby raises the likelihood of mistakes being associated therewith.

It could therefore be helpful to provide an ideally atraumatic, knotless or self-fixating wound closure system which, in respect of its construction and more particularly its handling, corresponds in principle to a surgical suture yet does not require additional auxiliary means for avoiding tissue trauma on introducing the wound closure system into a biological tissue.

### SUMMARY

We provide an elastomeric thread for use as a knotless or self-fixating wound closure system including on its surface anchoring structures for anchoring in biological, more particularly human and/or animal, tissues.

We also provide a wound closure system including the elastomeric thread.

We further provide a surgical kit of parts including the elastomeric thread and at least a surgical inserting instrument.

We still further provide a process for producing the elastomeric thread, including forming the anchoring structures on the surface of the elastomeric thread.

### BRIEF DESCRIPTION OF THE DRAWINGS

FIG. 1A is a side view of an example of our thread with the barbs in a relaxed longitudinal position.

FIG. 1B is a side view of our thread with barbs in a partially erect position.

FIG. 1C is a side view of our thread with barbs in a fully erect position.

### DETAILED DESCRIPTION

Our thread comprises an elastomeric thread, preferably for use as a knotless or self-fixating wound closure system, more particularly in the manner of a knotless or self-fixating surgical suture, wherein the thread includes on its surface anchoring structures for anchoring in biological, more particularly human and/or animal, tissues.

In other words, we provide a thread based on an elastomeric material, generally a polymer having elastomeric or elastic properties (a so-called “elastomer”), wherein anchoring structures are formed on the thread surface for anchoring the thread in biological tissues.

The biological tissues may in principle comprise hard and/or soft tissues. The tissues are preferably selected from the group consisting of skin, fat, fascia, bone, muscle, organs, nerves, blood vessels, connective tissue, sinews, tendons and ligaments. The thread is generally useful for tissue fixation and/or tissue lifting. The elastomeric thread is more particularly suitable for applications in plastic surgery, preferably for tightening the skin. For example, the elastomeric thread is suitable for performing an eyebrow lift. Further fields of application are cheek and/or jaw line corrections. In addition, however, the thread is also suitable for other surgical indications, more particularly for indications where the use of conventional sutures is made difficult by steric hindrance, for example. The elastomeric thread is thus particularly useful for performing laparoscopic interventions. A further field of use concerns the fixation of implants, more particularly textile implants. Examples of textile implants include hernia, prolapse and urinary incontinence meshes. A further possible field of application for the thread relates to the performance of anastomoses, in particular vascular or intestinal anastomoses.

Preferably, the anchoring structures absent an axial pulling load on the thread (relaxed or nonelongated state of the thread) essentially do not protrude from the thread surface so that the thread has an essentially smooth surface. As a result, tissue traumas on pulling the thread through a biological tissue can be substantially avoided. The expression “essentially smooth” as understood herein includes minor elevations on the thread surface capable of resulting from a position of the anchoring structures close to the thread surface in the nonelongated state of the thread. Ideally, however, the thread surface is completely smooth in the relaxed or nonelongated state of the thread.

The anchoring structures themselves are preferably erectable, more particularly reversibly erectable. The anchoring structures are preferably erectable on imposition of an axial pulling load on the thread, i.e., by applying a pulling force in the longitudinal direction of the thread, preferably at its ends. When the thread is extended or elongated by application of an axial pulling force, the anchoring structures become erect with particular advantage. The anchoring structures become erect because the force applied, although extending the thread by simultaneously reducing the thread diameter, does not transmit, or transmits at least only partially, to the anchoring structures. On removal of the pulling force, the thread relaxes, i.e., shortens, again as a consequence of the elastic restoring force, whereby the anchoring structures anchor themselves in the tissue and can, for example, approximate wound edges or tighten skin in cosmetic surgery.

Preferably, the anchoring structures in an erected state form an angle between 10 and 80°, more particularly 15 and 70° and preferably 20 and 60°, with the surface of the thread. Further, the anchoring structures absent an axial pulling load on the thread, i.e., in the relaxed state of the thread, may be formed on the thread surface in a density between 1 and 30 and more particularly 4 and 20 per cm of thread length (projected in intervals onto the longitudinal axis of the thread).

The thread comprises in general a thread main body, on the surface of which the anchoring structures for anchoring in biological, more particularly human and/or animal, tissues are formed. The thread main body usually has an elongate form. Preferably, the thread main body and the anchoring structures are formed in one piece. In principle, the thread main body and the anchoring structures can be formed from different elastomeric materials, more particularly elastomers. Preferably, the anchoring structures are formed from the same elastomeric material, more particularly the same elastomer, as the thread main body. The thread main body may be formed from an elastomeric material, more particularly an elastomer, and the anchoring structures are formed from a nonelastomeric material, more particularly nonelastomeric polymer. With regard to possible elastomeric materials, more particularly elastomers, the following description is referenced in full.

The thread can in principle be made of all elastomeric materials suitable therefor. Preferably, the thread material comprises a polymer having elastomeric properties, i.e., an elastomer. The polymer can be a homo-, co-, ter- or tetrapolymer and so on. In what follows, the term “copolymers” is to be understood as generally meaning polymers composed of two or more different monomeric units. In other words, useful copolymers may also comprise terpolymers, tetrapolymers, and so on. Suitable elastomers are more particularly formed as block polymers or segmented polymers. Formation as random or adventitious or alternating co-, ter- or tetrapolymers and so on is similarly possible. It is particularly preferable when the thread is formed from a block copolymer, block terpolymer, segmented polymer or a polymer blend. The block co- or block terpolymer may more particularly comprise a multiblock copolymer and a multiblock terpolymer, respectively. More particularly, the thread may be formed from a thermoplastic elastomer. Thermoplastic elastomers generally comprise plastics which at room temperature behave comparably to the classic elastomers, but on heating are plastically deformable and thus exhibit a thermoplastic behavior. Ideally, thermoplastic elastomers have a transition temperature to the plastically deformed behavior which is sufficiently above the body temperature of a patient. The use of thermoplastic elastomers has the advantage that they are significantly better to process than the classic elastomers. Classic elastomers are generally chemically wide-meshedly crosslinked spatial net molecules wherein the crosslinks can generally not be broken without decomposition of the elastomeric material. By contrast, thermoplastic elastomers include subregions of physical points of crosslinking, which can be broken on heating without the thermoplastic elastomeric material decomposing as a result.

The elastomeric thread may be formed from nonabsorbable, partially absorbable or fully absorbable elastomers. Nonabsorbable elastomers are preferably selected from the group consisting of thermoplastic elastomers based on olefin, thermoplastic elastomers based on urethane, or thermoplastic polyurethanes, thermoplastic copolyesters, styrene block copolymers, thermoplastic copolyamides, silicone elastomers, copolymers thereof and mixtures thereof. For example, the thread can be formed from a nonabsorbable elastomer from the group consisting of acrylic rubber, polyester-urethane rubber, brominated butyl rubber, chlorinated butyl rubber, chlorinated polyethylene, epichlorohydrin homopolymer, polychloroprene, sulphonated polyethylene, ethylene-acrylate rubber, epichlorohydrin copolymer, sulphur-crosslinked ethylene-propylene-diene rubber, peroxidically crosslinked ethylene-propylene copolymer, polyether-urethane rubber, ethylene-vinyl acetate copolymer, fluororubber, fluorosilicone rubber, hydrogenated nitrile rubber, butyl rubber, vinyl-containing dimethylpolysiloxane, nitrile rubber, butadiene-acrylonitrile rubber, natural rubber, thioplasts, polyfluorophosphazenes, polynorbonene, styrene-butadiene rubber, carboxyl-containing nitrile rubber, copolymers thereof and mixtures thereof. The thermoplastic polyurethanes mentioned in this section may in principle comprise aliphatic or aromatic polyurethanes. The polyurethanes may more particularly be branched or unbranched. Unbranched aliphatic thermoplastic polyurethanes are preferred. The thermoplastic polyurethanes may be selected from the group consisting of aliphatic polycarbonate urethanes, aromatic polycarbonate urethanes, silicone polycarbonate urethanes, silicone polyether urethanes and combinations thereof. The polycarbonate urethanes are generally prepared starting from a polycarbonate having terminal hydroxyl groups, an aromatic diisocyanate and also an oligoglycol or polyglycol.

The elastomeric thread may be composed of absorbable elastomers selected from the group consisting of polyhydroxybutyrates, block copolymers thereof, block terpolymers thereof and combinations, more particularly blends, thereof. Suitable block copolymers comprise glycolide, lactide, ε-caprolactone, trimethylene carbonate, p-dioxanone, 3-hydroxybutyrate and/or 4-hydroxybutyrate. Advantageous block terpolymers comprise glycolide, lactide, ε-caprolactone, trimethylene carbonate, p-dioxanone, 3-hydroxybutyrate and/or 4-hydroxybutyrate. For example, the thread can be formed from a triblock terpolymer comprising glycolide, trimethylene carbonate and 8-caprolactone. Such a triblock terpolymer is commercially available, for example, under the name of Monosyn®. The aforementioned polyhydroxybutyrates preferably comprise poly-3-hydroxybutyrate and/or poly-4-hydroxybutyrate.

The anchoring structures and the thread surface may be formed in one piece. The anchoring structures are preferably formed from the elastomeric thread material. The anchoring structures are preferably formed as cuts into the surface of the elastomeric thread. The cuts may comprise mechanical, physico-chemical, more particularly laser-produced, or thermal cuts. The cuts may have a cut depth, measured perpendicularly from the thread surface, between 5 and 60%, more particularly 15 and 50%, preferably 20 and 40%, based on the diameter of the thread.

The anchoring structures on imposition of an axial pulling load on the thread may form a barb, escutcheon, shield, scale, wedge, spike, arrow, V and/or W shape. It is particularly preferable for the anchoring structures to be designed in the manner of barbs or as barbs.

The anchoring structures may on imposition of an axial pulling load on the thread be in principle formed in different arrangements on the thread surface. For example, the anchoring structures may in this case have a row-shaped arrangement, a staggered arrangement, a zigzag-shaped arrangement, a spiral-shaped arrangement, a helical-shaped arrangement, a random arrangement or combinations thereof in the longitudinal and/or transverse direction, preferably in the longitudinal direction, of the thread.

The elastomeric thread on imposition of an axial pulling load may include at least one set, more particularly two, three or more sets, of anchoring structures. A set of anchoring structures is herein to be understood as meaning an arrangement of anchoring structures on the thread which in respect of the configuration of the anchoring structures, for example, length of the anchoring structures, height of the anchoring structures, cut depth of the anchoring structures, angle of the anchoring structures which the anchoring structures form in the erected state with the thread surface, orientation of the anchoring structures and/or shape or form of the anchoring structures coincides.

The anchoring structures on imposition of an axial pulling load on the thread may form a unidirectional arrangement on the surface of the thread. In other words, the anchoring structures point uniformly in one direction.

Preferably, the elastomeric thread on imposition of an axial pulling load has a so-called “bidirectional arrangement” of the anchoring structures on the thread surface. A bidirectional arrangement of anchoring structures is herein to be understood as meaning an arrangement wherein the anchoring structures are formed in two different directions (bidirectionally) on the thread surface. Preferably, viewed in the longitudinal direction of the thread, the anchoring structures point for a first thread portion in the direction of a remaining second thread portion and for the remaining second thread portion point in the direction of the first thread portion. For example, viewed in the longitudinal direction of the thread, the anchoring structures for a first thread portion may be formed in the direction of the thread middle and for a remaining second thread portion may likewise be formed in the direction of thread middle. The length of the thread portions may correspond approximately to half the thread length.

The elastomeric thread on imposition of an axial pulling load may display on its surface at least two bidirectional arrangements of anchoring structures. It is particularly preferable when, relative to a first bidirectional arrangement of anchoring structures, a second bidirectional arrangement of anchoring structures is formed on the thread surface in the circumferential direction of the thread by about 180° and preferably offset to the first bidirectional arrangement. It can further be provided for the elastomeric thread to display altogether three bidirectional arrangements of anchoring structures on imposition of an axial pulling load. It is preferable in this case when, relative to a first bidirectional arrangement of anchoring structures, a second bidirectional arrangement of anchoring structures is formed on the thread surface in the circumferential direction of the thread by about 120° and preferably offset to the first bidirectional arrangement, and this second bidirectional arrangement of anchoring structures being in turn formed in the circumferential direction of the thread by about 120° and preferably offset relative to a third bidirectional arrangement of anchoring structures, so that the third bidirectional arrangement of anchoring structures is similarly formed in the circumferential direction of the thread by about 120° and preferably offset relative to the first bidirectional arrangement of anchoring structures.

Preferably, the anchoring structures on imposition of an axial pulling load on the thread have a periodically changing, more particularly alternating, orientation on the thread surface. For example, viewed in the longitudinal direction of the thread, the anchoring structures for a first thread portion can be formed in the direction of a second thread portion and for the second thread portion in the direction of the first thread portion, for a third thread portion adjoining the second thread portion in the direction of a fourth thread portion and for the fourth thread portion in the direction of the third thread portion, and so on.

It can further be provided for the elastomeric thread to display areal regions or areal portions without anchoring structures. Preferably, the thread displays an areal portion without anchoring structures approximately in the region of the thread middle. This areal portion, viewed in the longitudinal direction of the thread, can have a length between 0.5 and 5 cm, more particularly 1.5 and 3 cm. This makes it possible to place the thread ends side by side to form a loop and to attach them to a surgical inserting instrument, preferably to a surgical needle. In this example, the remaining areal portions of the thread preferably display a bidirectional arrangement of anchoring structures, so that, after loop formation, the anchoring structures point unidirectionally in the direction of the loop. Since the thread middle generally is used for the actual wound closure, a thread whose thread middle is essentially free of anchoring structures can be used with particular advantage to avoid additional irritations of tissue in the wound region. With regard to the arrangement possibilities for anchoring structures on the thread surface on imposition of an axial pulling load on the thread, reference is made to the description heretofore.

The anchoring structures may include a coating, preferably a stiffening coating. The coating is preferably only formed in the region of the anchoring structures, more particularly exclusively on the anchoring structures. The coating may only be formed partially on the anchoring structures. For example, the coating may only be formed on the reverse side of the anchoring structures. Furthermore, the coating may only be formed in the region of the ends protruding from the thread surface, preferably only on the tips, of the anchoring structures, more particularly on both sides thereof. However, it is preferable for the coating to surround the anchoring structures in full, i.e., for the surface of the anchoring structures to be completely covered with the coating. The coating preferably comprises a proportion between 0.5% and 25% by weight, more particularly 1% and 20% by weight and preferably 2% and 15% by weight, based on the total weight of the thread. In general, the layer thickness of the coating is chosen such that the coated anchoring structures still protrude from the thread surface. The layer thickness of the coating is preferably between 1 and 100 μm, more particularly 2 and 50 μm and more preferably 4 and 40 μm. In general, the coating is formed from a different material than the thread per se. Preferably, the coating includes a biocompatible material, more particularly polymer, having a flexural modulus>1500 N/mm², preferably >4000 N/mm². More particularly, the coating is formed from such a material, more particularly polymer.

The coating which is preferably a stiffening coating may further be formed to be fully absorbable, partially absorbable or nonabsorbable. More particularly, the stiffening coating may include an absorbable polymer and more particularly be formed from such. Useful polymers include in particular polyhydroxyalkanoates, copolymers thereof and combinations thereof. The coating preferably includes an absorbable polymer selected from the group consisting of polylactide, polyglycolide, poly-ε-caprolactone, polytrimethylene carbonate, poly-p-dioxanone, copolymers thereof and combinations, more particularly blends, thereof. Preferably the coating is formed from one of the polymers mentioned in this section, copolymers mentioned in this section or from one of the combinations mentioned in this section, more particularly blends, thereof.

Alternatively, the coating, preferably stiffening coating, includes a nonabsorbable polymer, more particularly selected from the group consisting of polyethylene terephthalate, polypropylene, polyvinylidene difluoride, polytetrafluoroethylene, polyhexafluoropropylene, polyamides, copolymers thereof and combinations, more particularly blends, thereof. Preferably, the coating is formed from a polymer or copolymer mentioned in this section or from one of the combinations, more particularly blends, mentioned in this section.

The thread may have a linear tensile strength between 30 and 500 N/mm², more particularly 50 and 350 N/mm², based on a thread without anchoring structures. The thread may further have an elongation at break between 50 and 700%, more particularly 80 and 600%, preferably 150 and 500%. The thread preferably has a flexural modulus of 3 to 300 N/mm², more particularly 5 to 200 N/mm².

The thread can be formed as a monofilament or as a multifilament, more particularly as a braided or intertwined multifilament. Preferably, the elastomeric thread is formed as a monofilament or as a monofil thread. It is further possible for the thread to be formed as a pseudomonofilament.

The thread may be designed as a mass or solid thread. As used here, the term “mass or solid thread” may, in particular, mean that the thread has no lumen.

Alternatively, the thread is designed as a hollow thread, in particular as a tubular thread, preferably as a tube or hose. The hollow thread may be, in particular, featured by a closed wall encasing a lumen, wherein the ends of the hollow thread are preferably open. The hollow thread may be produced by means of extrusion. Further, the hollow thread may have an internal diameter<1 cm, in particular <5 mm, preferably <2 mm. The anchoring structures, on imposition of an axial pulling load on the hollow thread, may be arranged into the interior and/or exterior, preferably exterior, of the hollow thread. The anchoring structures may be cut into the wall of the hollow thread, wherein the cuts preferably do not break through the wall of the hollow thread, and/or may be present as breakthroughs, i.e., the anchoring structures completely break through the wall of the hollow thread.

Hollow threads may be employed in various fields of medicine. For instance, the hollow threads may be used as a self-anchoring infusion tubes, delivery tubes, catheters, distribution systems for medicaments, in particular liquid medicaments, drug-release-systems or drainage systems, in particular drainage tubes. Besides, the hollow threads may be applied, in particular shrunk and/or drawn, on a round stock, fibre, monofilament, pseudomonofilament, multifilament, yarn, thread or the like to equip these structures with self-anchoring properties.

The thread generally has a circular cross section. However, other cross-sectional shapes are conceivable as well. For example, the thread may have an oval, triangular, trilobal, square, trapezoidal, rhomboid, pentagonal/five-cornered, hexagonal/six-cornered, star-shaped or cruciform cross section. Such cross-sectional shapes are readily realizable with the aid of appropriate extrusion dies which can be custom made to any desired cross-sectional shape.

The elastomeric thread includes added substances, more particularly biological and/or medical actives. For example, the thread may include antimicrobial, disinfecting, growth-promoting, anti-inflammatory, blood-coagulating, pain-killing (analgesic) and/or odor-controlling actives.

The elastomeric thread may be present in the form of a continuous-filament thread, in particular continuous-filament fiber, or as a converted, more particularly cut to length, fiber.

The elastomeric thread may be fitted at least at one end, preferably at both ends, with a surgical inserting instrument. The surgical inserting instrument preferably comprises a surgical needle. The use of a canula as inserting instrument is similarly possible. Preferably, at least one end of the canula has been sharpened with an oblique grind such that a small cut be made on penetration into a tissue. Alternatively, the canula may also have a conically tapered and more particularly pointed end. In the case of the above-described loop-shaped thread, it is generally the case that both ends of the thread are attached to a surgical inserting instrument. However, when the thread has a bidirectional arrangement of anchoring structures, it is preferable when both of the ends of the thread are each connected to a surgical inserting instrument. To connect the thread to a surgical inserting instrument, the thread can be inserted into a dedicated drilled hole in the inserting instrument and the inserting instrument subsequently be compressed or beaded in the region of the drilled hole.

To avoid puncture channel bleeds, it can be further provided for the thread to have a smaller diameter in the region of its ends than in the remaining thread regions. In other words, the ends of the thread may have a tapered diameter. Such a thread is particularly advantageously combined with a surgical inserting instrument, for example, a surgical needle, that is actually designed for a smaller thread diameter. In this way, the thread diameter can be conformed to the diameter of the inserting instrument. Especially a diameter ratio of surgical inserting instrument to thread of less than 2:1, preferably of 1:1, can be provided. As a result, a puncture channel formed by the surgical inserting instrument can be better filled out by the thread regions having the original, i.e., untapered, diameter. A puncture channel whose diameter is very close to the diameter of the thread is advantageous in ensuring better anchoring of the thread in the tissue. To taper the diameter, the thread can be shaved in the region of its ends. Thermal methods or laser techniques can be used for this, for example. The transition from the original diameter of the thread to the tapered diameter in the region of the thread ends can be abrupt or continuous, more particularly in the form of a gradient. To form a gradual transition, extrusion technology is suitable in particular. The take-off speed involved in extruding a thread can be varied, more particularly periodically. This can be accomplished, for example, by modulating the circumferential speed of the godet responsible for the take-off of the thread. Alternatively, additional godets can be interposed between the extrusion die and the take-off godet.

The thread may be fixed in its lengthened or elongated state in the lumen of a blunt end of a canula. This results in a small ratio of outer diameter of the canula to diameter of the thread. Preferably, the canula is longer than the pull-through distance through the tissue. This can be realized, for example, by temporarily fixing the elastomeric thread in the elongated state (with tapered diameter) by freezing and then pushing it into the canula. As it thaws, the thread then becomes anchored in the canula as it shortens and its diameter increases, resulting in an optimal needle/thread diameter ratio.

The elastomeric thread may be present in a drawn or undrawn state.

We further provide a wound closure system or a surgical fixating means, more particularly a knotless or self-fixating wound closure system or surgical fixating means, preferably in the manner of a surgical suture, more particularly knotless or self-fixating surgical suture, comprising an elastomeric thread. The wound closure system is generally present as a thread-shaped wound closure system. As such, the wound closure system is preferably formed to be monofil. In principle, however, the wound closure system can also be formed to be multifil. In respect of further features and details concerning the wound closure system, reference is made to the description heretofore.

We further provide a surgical kit of parts comprising an elastomeric thread or a wound closure system and at least a surgical inserting instrument. As mentioned, the surgical inserting instrument may comprise a surgical needle or a canula. With regard to further features and details concerning the kit of parts, reference is likewise made to the description heretofore.

We further provide a process for producing a thread, wherein anchoring structures are formed on the surface of an elastomeric thread.

Preferably, the anchoring structures are formed by cutting into the thread surface.

The anchoring structures may be cut into the thread mechanically, preferably by at least one cutting blade, for example, a microtome knife or, as an alternative, by a needle, in particular a surgical needle, to less affect the mechanical stability of the thread. Customary cutting devices can be used for this purpose. Useful cutting devices generally comprise a cutting board, at least one cutting blade and also holding or fixing elements for the thread, for example, a vice, chucks, holding or clamping jaws. For example, cutting the anchoring elements mechanically may utilize a cutting board (cutting abutment) having a groove, the groove being intended to receive the thread to be cut into. Depending on the depth of the groove, the use of at least one cutting blade allows specific control of the cut depth to which the anchoring structures are cut into the thread. This is because the at least one cutting blade is generally configured such that with it only at most the regions of the thread which protrude from the groove can be cut into. This contributes with particular advantage to enhancing the cutting consistency in the process.

Alternatively, the anchoring structures are cut into the thread thermally, preferably in a temperature range between 10 and 100° C., more particularly 20 and 50° C., above the melting point of the thread material. Thermal cutting of the anchoring structures has the profound advantage over mechanical cutting that the cut ends in the thread which are produced by thermal cutting are less tapered, more particularly less acute, than results from purely mechanical cutting. This can be used to minimize the risk of the thread developing, under load, a tear starting from the respective cut ends. The anchoring structures may be cut into the thread by a cutting wire, more particularly metal wire, suitable for this purpose. A heated, more particularly an electrically heated, cutting wire can be used, for example. The cutting wire preferably comprises a fine wire. Preference is given to using a cutting wire having a diameter between 20 and 50 μm. As an alternative to a single cutting wire, it is also possible to use a sheet of cutting wires. It is similarly possible to use a metal grid.

Further alternatively, the anchoring structures are cut into the thread by laser cutting techniques. Useful lasers include not only gas lasers, for example, CO₂ lasers, but also solid state lasers, for example, Nd:YAG lasers. In general, a suitable laser cutting machine consists of a laser beam source, a beam guide and a usually mobile system of focusing optics (concave mirror or positive lens). The beam leaving the beam source is guided either through an optical fiber in the case of an Nd:YAG laser, for example, or via a deflecting mirror in the case of a CO₂ laser, for example, to the machining optics which focus the laser beam and thereby produce the power densities required for cutting, which generally range from 10⁶ to 10⁹ W/cm². Appropriate laser cutting processes are known, so that more far-reaching observations can be dispensed with here.

Particularly preferably, the anchoring structures are coated with a material, preferably a stiffening material, more particularly a stiffening polymer. Preferably, the thread is extended/elongated by application of an axial pulling force, preferably at the thread ends, to coat the anchoring structures. The anchoring structures, which are generally not subject to any significant extension or elongation, are preferably completely coated with the material and thereby stiffened. The anchoring structures can be coated by dipping into a coating solution or by deposition of a material from the gas phase. As a result, relatively long anchoring structures, which in contradistinction to relatively short anchoring structures generally have a higher flexural slackness, can be stiffened in particular, whereby their anchoring performance can be improved overall. It is particularly preferable when the thread is alternatingly elongated (extended) and relaxed until the stiffening material has cured. This measure makes it possible to prevent the elastic properties of the thread main body (thread body without the barbs) being impaired or lost entirely.

Cutting the anchoring structures can be effected in the drawn or undrawn state of the thread. Any drawing is preferably effected while heat is applied. To produce a heat advantageous for the drawing operation, warm water or infrared radiation can be used, for example. It is similarly possible for drawing to be carried out in a heating oven suitable for this purpose. To draw the thread, it is usually guided over a roller or godet system comprising a set of rollers or godets which can have different speeds of rotation. Generally, each subsequent roller has a higher speed of rotation than the preceding roller of the drawing system. However, alternatively to the just-described continuous drawing, it is also possible to carry out discontinuous drawing. For discontinuous drawing, the thread can be clamped between the clamping jaws of a clamping device and subsequently drawn.

After drawing, optionally also independently of any drawing, of the thread it is possible for the thread to be subjected to various post-treatment steps. In general, the thread is for this heat-conditioned in vacuo or at reduced pressure. This can be used to raise the crystallinity of the thread and, more particularly, lower the residual monomer content. A further advantage which can be achieved through a post-treatment of the thread relates to the reduced susceptibility to shrinking.

To minimize the righting angle of the anchoring structures, the thread may be subjected to relaxation, in particular shrinkage, subsequent to drawing. This is particularly advantageous with respect to polymers having a high drawing ratio. Such polymers may otherwise contribute to anchoring structures which may protrude perpendicularly from the thread surface on imposition of an axial pulling load on the thread. Further, due to relaxation, elasticity of the thread may be improved which is advantageous in view of handling properties and a more equal transfer of force on a tissue to be treated.

The thread may be subjected to relaxation, in particular shrinkage. Subsequently, the anchoring structures are cut into the relaxed thread. Thereafter, the cut-in thread is drawn. Thus, the righting angle of the anchoring structures may also be minimized.

We further provide for the use of the thread for producing a wound closure system or surgical fixating means, more particularly a knotless or self-fixating wound closure system or fixating means, preferably in the manner of a surgical suture, more particularly in the manner of a knotless or self-fixating surgical suture. The wound closure system or surgical fixating means is preferably useful for tissue fixation or tissue lifting. With regard to further features and details, the description heretofore is referred to in its entirety.

The thread may be used in the production of a fixating means for implants, more particularly textile implants. With regard to further features and details, the description heretofore is similarly referenced in full.

Finally, we provide a process for approximating, fixating and/or gathering together of biological, more particularly human and/or animal, tissue, comprising the steps of:
a) introducing into the tissue an elastomeric thread displaying on its surface anchoring structures for anchoring in biological, more particularly human and/or animal, tissues,
b) applying an axial pulling force to the inserted thread, more particularly at its ends, and
c) reducing, more particularly removing, the axial pulling force.

The elastomeric thread is particularly useful as a knotless or self-fixating wound closure system or surgical fixating means by displaying anchoring structures formed in an elastomeric thread material. When the thread is in a relaxed, i.e., nonextended, state, the anchoring structures combine with the remaining regions of the thread to form a preferably smooth surface for the thread. When the thread is then pulled through a biological, generally human or animal, tissue, this is preferably done by applying a force smaller than the force needed to erect the anchoring structures. This is particularly advantageous in completely avoiding tissue traumas. If desired, however, it is also possible for the thread to be inserted into a biological tissue using a force which leads to minimal erection or deployment of the anchoring structures. The tissue traumas caused by this are then still significantly less than when the thread is introduced into a biological tissue with the anchoring structures maximally erected or deployed. Once the thread is optimally placed in the eyes of the surgeon, the anchoring structures are erected by exerting a pulling force on the two ends of the thread. After removal of the pulling force from the ends of the thread, the thread shortens, and the anchoring structures anchor themselves and hence the thread in the tissue. Since, however, the thread does not shorten to its original length and therefore is still under a certain tension, the tissue is, more particularly, the wound edges are, approximated, fixated and/or gathered together. Since the anchoring structures only erect on imposition of a certain pulling load on the thread in its longitudinal direction (axial pulling load), no additional aids are needed to introduce the thread into a biological tissue. This renders the handling of the thread simple and free of complications. It is a further advantage of the thread that it can in practice be used as a knotless or self-fixating surgical suture. Since surgeons are sufficiently familiar with such sutures, this results in a further advantage for the practical handling of the thread.

Further features become apparent from the following description by reference to examples and a FIGURE description and the Drawing. Individual features can each be actualized on their own or two or more at a time in combination with each other.

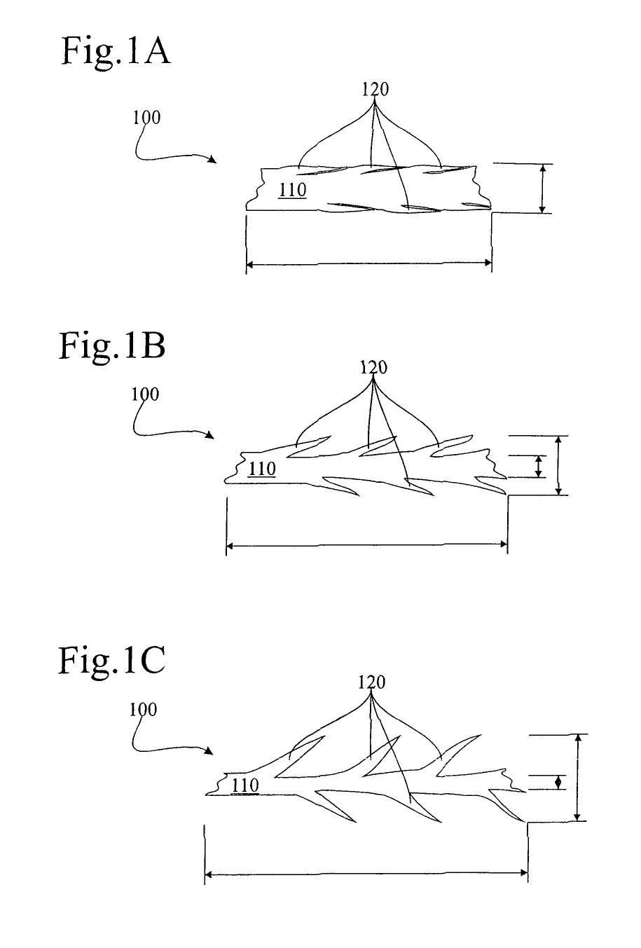

The Drawing depicts an example of our thread 100. The thread 100 has a thread main body 110 having anchoring structures 120 formed on its surface. In the relaxed (nonextended or nonelongated) state, the anchoring structures 120 combine with the thread main body 110 to form a preferably smooth surface for the thread (FIG. 1A). In this state, the thread can be pulled with particular advantage through biological tissue without significant tissue traumas occurring as a result. Advantageously, the thread can also be moved in biological tissue in a direction contrary to the potential blocking direction. When the thread 100 is subject to the imposition thereon of a certain pulling force in the longitudinal direction of the thread 100, preferably on the thread's ends, the anchoring structures 120 become erect (FIGS. 1B and 1C). Application of the pulling force reduces the diameter of the thread main body. However, the pulling force is transmitted only partially, if at all, to the anchoring structures 120. When the thread 100, therefore, is used as a surgical suture, the anchoring structures 120—after correct placement of the thread 100 in a biological tissue—can be erected by applying an axial pulling force to thereby establish contact with the surrounding region of tissue. Removing the axial pulling force causes the anchoring structures to become anchored in the biological tissue, since the anchoring structures are by virtue of the lengthwise contraction of the thread (due to the removal of the axial pulling force) pulled laterally of the thread 100 into the tissue. The elastic pull stops the anchoring structures becoming free again. As a result, the tissue can be approximated, fixated and/or gathered together as desired. In other words, an active approximation, fixation and/or gathering together of the tissue rests on the elastic restoring force of the thread 100.

### EXAMPLES

Production of Monofilaments from a Thermoplastic Elastomer (Vasomer)

Vasomer is a linear aliphatic polyurethane and a thermoplastic elastomer which is approved for medical implants and has a Shore A hardness of 76. Vasomer was processed into monofilaments on a TW 100 extruder (twin-screw extruder with Rheocord 90 drive from Haake). The cooled feed zone was followed at the extruder zones by a temperature profile rising from 160° C. for zone 1 to 200° C. at the spin head. The spin head was equipped with a 1.25 mm die with L/D 8. Mass throughput was merely set via the screw speed. To solidify the extruded strand following an air passage of 7 cm it was pulled through a cold water bath at 20° C. and subsequently wound up on a drum. Altogether, monofilaments were produced in two sizes. These are reproduced below in Table 1.

TABLE 1

| Monofilament | MV1 | MV2 |
|---|---|---|
| Screw speed [rpm] | 24 | 22 |
| Die pressure [bar] | 45 | 40 |
| Winding speed [m/min] | 13.0 | 6.4 |
| Diameter [mm] | 0.80 | 1.05 |

The flexural modulus of the monofilaments, as measured on a Frank 58963 flexural stiffness tester at a clamped length of 5 mm and a bending angle of 30°, was merely 6 N/mm².

Stress-strain measurements and also hysteresis tests concerning reversible elasticity were carried out on a Zwick 1455 tensile tester. The maximum force for MV2 was found to be 39.1 N. The related extension was 410%±10%. Since the original diameter was approximately halved at elongation around 400%, strengths close to the USP requirements for surgical sutures were obtained. Hysteresis tests at 200% and 300% showed virtually complete reversibility.

Production of a Vasomer Monofilament with Barbs

The cutting of MV2 from the example above was done on a specially designed prototype cutting machine. The cutting device used was a microtome knife, which cut into the MV2 monofilament pneumatically and at controllable time intervals. The cutting abutment and transporter used was a cutting wheel which was equipped with a fine guiding groove and onto which the Vasomer monofilament was clamped or adhered under most minimal elongation. The cutting wheel itself was driven at a controllable speed. The cut length, the cut angle and the resulting cut depth (perpendicularly to the monofilament surface) were likewise able to be varied. The results reproduced below in Table 2 were obtained.

TABLE 2

| | Sample | | | | | |
|---|---|---|---|---|---|---|
| | Vas B1 | Vas B2 | Vas B3 | Vas B4 | Vas B5 | Vas B6 |
| State | elastic, non- | elastic, non- | elastic, non- | elastic, non- | elastic, non- | elastic, non- |
| | elongated | elongated | elongated | elongated | elongated | elongated |
| Blade | Microtome | Microtome | Microtome | Microtome | Microtome | Microtome |
| Cut length | 0.2 | 0.45 | 0.7 | 1.05 | 0.3 | 0.7 |
| [mm] | | | | | | |
| Perpendicular | 0.07 | 0.15 | 0.24 | 0.36 | 0.15 | 0.35 |
| cut depth | | | | | | |
| [mm] | | | | | | |
| Angle [°] | 20 | 20 | 20 | 20 | 30 | 30 |

In the nonelongated state (even after repeated reversible elongation), the cuts were virtually not noticeable. The barbs in this state thus did not protrude from the monofilament surface. On the contrary, the barbs only deployed on elongation due to a pulling load in the longitudinal direction of the monofilament. After removal of the pulling tension and the return of the monofilament in its original state, the barbs became fully closed again and combined with the monofilament main body to form a completely smooth surface.

These tests document that thread is very useful to be inserted virtually atraumatically into biological tissue using a suitable inserting instrument, generally a surgical needle, while in the nonelongated or only minimally elongated state. Application of a pulling load between the two ends of the thread outside the tissue renders the anchoring structures, formed as barbs in the above examples, erectable or deployable. By subsequent gradual withdrawal of the pulling load, the inserted thread starts with a length reduction by closing the anchoring structures, causing these to anchor themselves in the tissue and at the same time to approximate and gather together the tissue, for example, wound edges, or facilitating lifting, for example.

1. An elastomeric thread for use as a knotless or self-fixating wound closure system comprising on its surface anchoring structures for anchoring in biological, more particularly human and/or animal, tissues.

2. The elastomeric thread according to claim 1, wherein the anchoring structures absent an axial pulling load on the thread essentially do not protrude from the thread surface so that the thread has an essentially smooth surface.

3. The elastomeric thread according to claim 1, wherein the anchoring structures are reversibly erectable on imposition of an axial pulling load on the thread.

4. The elastomeric thread according to claim 1, wherein the anchoring structures in an erected state form an angle between 10 and 80° with the surface of the thread.

5. The elastomeric thread according to claim 1, wherein the anchoring structures absent axial pulling load on the thread are formed on the surface at a density of 1 to 30 per cm of thread length.

6. The elastomeric thread according to claim 1, formed from a block copolymer, block terpolymer, segmented polymer or polymer blend.

7. The elastomeric thread according to claim 1, formed from a thermoplastic elastomer.

8. The elastomeric thread according to claim 1, formed from nonabsorbable elastomers selected from the group consisting of thermoplastic elastomers based on olefin, thermoplastic elastomers based on urethane, thermoplastic polyurethanes, thermoplastic copolyesters, styrene block copolymers, thermoplastic copolyamides, silicone elastomers and mixtures thereof.

9. The elastomeric thread according to claim 1, formed from absorbable elastomers selected from the group consisting of polyhydroxybutyrates, block copolymers comprising gly-colide, lactide, ε-caprolactone, trimethylene carbonate, p-dioxanone, 3-hydroxybutyrate and/or 4-hydroxybutyrate, and block terpolymers comprising glycolide, lactide, ε-capro-lactone, trimethylene carbonate, p-dioxanone, 3-hydroxybutyrate and/or 4-hydroxy-butyrate.

10. The elastomeric thread according to claim 1, wherein the anchoring structures are formed as cuts into the surface of the thread.

11. The elastomeric thread according to claim 1, wherein the anchoring structures have a cut depth, measured perpendicularly from the thread surface, between 5 and 60%, based on the diameter of the thread.

12. The elastomeric thread according to claim 1, wherein the anchoring structures on imposition of an axial pulling load on the thread form a barb, escutcheon, shield, scale, wedge, spike, arrow, V and/or W shape on the surface of the thread.

13. The elastomeric thread according to claim 1, wherein the anchoring structures on imposition of an axial pulling load on the thread form a unidirectional arrangement on the surface of the thread.

14. The elastomeric thread according to claim 1, wherein the anchoring structures on imposition of an axial pulling load on the thread form a bidirectional arrangement on the thread surface.

15. The elastomeric thread according to claim 1, wherein the anchoring structures on imposition of an axial pulling load on the thread point for a first thread portion in a direction of a remaining second thread portion and for the remaining second thread portion point in a direction of the first thread portion.

16. The elastomeric thread according to claim 1, wherein the anchoring structures on imposition of an axial pulling load on the thread have an alternating orientation on the thread surface.

17. The thread according to claim 1, wherein the anchoring structures are coated with a stiffening polymer.

18. The elastomeric thread according to claim 1, having a linear tensile strength between 30 and 500 N/mm².

19. The elastomeric thread according to claim 1, having an elongation at break between 50 and 700%.

20. The elastomeric thread according to claim 1, fitted with a surgical inserting instrument at least at one end.

21. The elastomeric thread according to claim 1, which is a mass thread.

22. The elastomeric thread according to claim 1, which is a tube.

23. A wound closure system comprising an elastomeric thread according to claim 1.

24. A surgical kit of parts comprising the elastomeric thread according to claim 1 and at least a surgical inserting instrument.

25. A process for producing the elastomeric thread according to claim 1, comprising forming the anchoring structures on the surface of the elastomeric thread.

26. The process according to claim 25, wherein the anchoring structures are formed by cutting into the thread surface.

27. The process according to claim 25, wherein the anchoring structures are coated with a stiffening polymer.

28. The process according to claim 27, wherein the thread is extended/elongated by application of an axial pulling force at ends of the thread to coat the anchoring structures.

29. The process according to claim 27, wherein the anchoring structures are coated by dipping into a coating solution or by deposition of a material from a gas phase.

30. The process according to claim 27, wherein the thread is alternatingly elongated and relaxed until the stiffening material has cured.

31. (canceled)

32. (canceled)
